(19) United States
(12) Patent Application Publication
(10) Pub. No.: US 2009/0017143 A1
(43) Pub. Date: Jan. 15, 2009

(54) Composition for treatment of alcoholism

(76) Inventor: Doug Krotzer, Jacksonville, FL (US)

Correspondence Address: ROGERS TOWERS, P.A., 1301 RIVERPLACE BOULEVARD, SUITE 1500, JACKSONVILLE, FL, 32207, US

(21) Appl. No.: 12/217,804

(22) Filed: Jul. 9, 2008

Related U.S. Application Data
(60) Provisional application No. 60/958,950, filed on Jul. 10, 2007.

(51) Int. Cl. A61K 36/38; A61K 36/488; A61P 25/32

(52) U.S. Cl. 424/725; 424/730

(57) ABSTRACT

A composition effective in the treatment of alcoholism comprising one or more alcohol craving blocker components such as Kudzu and Rhodiola Rosea, one or more antidepressant components such as St. John's Wort (Hypericin), 5-Hydroxytryptophanm SAMe, Melatonin and Taurine, and/or one or more anti-anxiety components such as Vitamin B complex, Glutamine, Niacin, SAMe, Hops, Melatonin and Rhodiola Rosea, the components chosen such that in combination they beneficially affect the nutritional, physiological and psychological deficiencies that combine to cause alcohol dependency.

This patent application claims the benefit of U.S. Provisional Patent application Ser. No. 60/958,950, filed Jul. 10, 2007.

### BACKGROUND OF THE INVENTION

This invention relates generally to the field of compositions used to treat addictive diseases, more particularly alcoholism, and more particularly relates to such compositions comprising nutraceuticals, i.e., a product isolated or purified from foods, and generally sold in medicinal forms not usually associated with food and demonstrated to have a physiological benefit or provide protection against chronic disease, as opposed to pharmaceuticals, i.e. synthesized drugs. The compositions have nutritionally beneficial components that address specific deficiencies and/or address other nutritional, physiological or psychological concerns (such as for example depression, anxiety, unstable blood sugar levels, energy levels or organ problems) that in combination result in alcohol dependency.

By the mid 1990's research had conclusively established that alcohol abuse and alcoholism is caused by a lack of neurotransmitters in the Nucleus Accumbens and Ventral Tegmental areas, also known as the “pleasure centers”, of the brain. Ten important neurotransmitters were identified, and their variants are now known to comprise over 50 distinct molecules. Every individual prone to alcoholism will be lacking in different neurotransmitters and in differing amounts of neurotransmitters, meaning that each individual requires a custom solution to replace the cravings that result in alcoholism.

It is an object of this invention to provide compositions to address the physiological deficiencies that result in alcohol dependency or addiction because of deficiencies in neurotransmitters within an individual, and it is a further object to provide compositions that also address related nutritional, physiological or psychological deficiencies that in combination with the neurotransmitter deficiencies increase the likelihood or severity of alcoholism in a particular individual.

### SUMMARY OF THE INVENTION

The invention is a composition effective in the treatment of alcoholism, the composition comprising a plurality of different components known as nutraceuticals (i.e., a product isolated or purified from foods), as opposed to pharmaceuticals (i.e., synthesized drugs). The composition has multiple embodiments such that a customized composition for a particular individual may be provided. The composition addresses neurotransmitter deficiencies and further addresses related nutritional, physiological and psychological conditions that adversely affect an individual's response to alcohol, such as depression, anxiety, unstable blood sugar levels, energy levels or organ problems.

In a basic embodiment, the composition comprises one or more alcohol craving blocker components, one or more antidepressant components, and/or one or more anti-anxiety components (i.e., anxiolytics), each component present in amount sufficient to beneficially minimize or obviate the nutritional, physiological and psychological conditions that in combination result in alcohol dependency. In addition to the above, alternative embodiments of the composition directed at specific needs will comprise individually or in combination blood sugar stabilizer components, energizer components and organ repair components.

The alcohol craving blocker component of the composition consists of one or more components chosen from the group of components consisting of Kudzu (Daidzin) and Rhodiola Rosea. The antidepressant component of the composition consists of one or more components chosen from the group of components consisting of St. John's Wort (Hypericin), 5-Hydroxytryptophanm SAMe, Melatonin and Taurine. The anxiolytic component of the composition consists of one or more components chosen from the group of components consisting of Vitamin B complex, Glutamine, Niacin, SAMe, Hops, Melatonin and Rhodiola Rosea. The blood sugar stabilizer component of the composition consists of one or more components chosen from the group of components consisting of Niacin and Chromium Picolinate. The energizer component of the composition consists of one or more components chosen from the group of components consisting of Glutamine and Taurine. The organ repair component of the composition consists of one or more components chosen from the group of components consisting of Glutamine, Milk Thistle (Silymarin), Niacin and Taurine. As noted, some of the component members address multiple deficiencies or problems.

### DETAILED DESCRIPTION OF THE INVENTION

The best mode and preferred embodiments of the invention are described below. In general, the invention is a composition containing multiple components that addresses the physiological deficiencies that result in alcohol dependency or addiction because of deficiencies in neurotransmitters within an individual, and that also addresses related nutritional, physiological or psychological deficiencies that in combination with the neurotransmitter deficiencies increase the likelihood or severity of alcoholism in a particular individual. The composition is preferably formulated to be taken orally.

In a basic embodiment, the composition comprises in combination efficacious amounts of nutritionally, physiologically and psychologically beneficial components that address alcohol craving, depression problems and/or anxiety problems. This basic embodiment therefore consists of one or more alcohol craving blocker components present in an amount effective to minimize or obviate alcohol craving, one or more antidepressant components present in an amount effective to minimize or obviate depression, and/or one or more anti-anxiety components (i.e., anxiolytics) present in an amount effective to minimize or obviate anxiety.

The alcohol craving blocker component of the composition consists of one or more components chosen from the group of components consisting of Kudzu (Daidzin) and Rhodiola Rosea. The antidepressant component of the composition consists of one or more components chosen from the group of components consisting of St. John's Wort (Hypericin), 5-Hydroxytryptophanm SAMe, Melatonin and Taurine. The anxiolytic component of the composition consists of one or more components chosen from the group of components consisting of Vitamin B complex, Glutamine, Niacin (true Niacin, not Niacinamide; which is often mistakenly referred to as Niacin), SAMe, Hops, Melatonin and Rhodiola Rosea. Vitamin B complex is taken herein to define a grouping of related components comprising one or more of the vitamins B1, B2, Niacinamide, B6, B12, Folic Acid, Biotin, Pantothenic Acid, Choline Bitartrate, Inositol and Para-aminobenzoic Acid.

Preferred compositions comprise the following combinations comprising the listed components:
(a) Kudzu and St. John's Wort
(b) Kudzu and Vitamin B complex
(c) Kudzu and St. John's Wort and Vitamin B complex
(d) Kudzu and 5-Hydroxytryptophan
(e) Kudzu and 5-Hydroxytryptophan and Vitamin B Complex
(f) Kudzu and Taurine
(g) Kudzu and Taurine and Vitamin B complex
(h) Kudzu and Melatonin
(i) Kudzu and Melatonin and Vitamin B complex

Daily effective dosage amount ranges for the components, the amounts being effective when given in combination as described to beneficially address the nutritional, physiological and psychological deficiencies that result in alcohol dependency and addiction, are as follows:

Kudzu: from about 450 to 11,500 mg, preferably from about 1,350 to 4,100 mg, and most preferably from about 2,300 to 3,200 mg;

St. John's Wort: from about 300 to 6,000 mg, preferably from about 600 to 4,700 mg, and most preferably from about 1,600 to 3,700 mg;

Vitamin B complex: for each component present, from about 5 to 500 mg B1, from about 5 to 500 mg B2, from about 5 to 500 mg Niacinamide, from about 5 to 500 mg B6, from about 5 to 500 mcg B12, from about 5 to 500 mcg Folic Acid, from about 5 to 500 mcg Biotin, from about 5 to 500 mg Pantothenic Acid, from about 5 to 500 mg Choline Bitartrate, from about 5 to 500 mg Inositol and from about 5 to 500 mg Para-aminobenzoic Acid; preferably from about 100 to 400 mg B1, from about 100 to 400 mg B2, from about 100 to 400 mg Niacinamide, from about 100 to 400 mg B6, from about 100 to 400 mcg B12, from about 100 to 400 mcg Folic Acid, from about 100 to 400 mcg Biotin, from about 100 to 400 mg Pantothenic Acid, from about 100 to 400 mg Choline Bitartrate, from about 100 to 400 mg Inositol and from about 100 to 400 mg Para-aminobenzoic Acid; and most preferably from about 200 to 300 mg B1, from about 200 to 300 mg B2, from about 200 to 300 mg Niacinamide, from about 200 to 300 mg B6, from about 200 to 300 mcg B12, from about 200 to 300 mcg Folic Acid, from about 200 to 300 mcg Biotin, from about 200 to 300 mg Pantothenic Acid, from about 200 to 300 mg Choline Bitartrate, from about 200 to 300 mg Inositol and from about 200 to 300 mg Para-aminobenzoic Acid;

5-Hydroxytryptophan: from about 25 to 800 mg, preferably from about 100 to 500 mg, and most preferably from about 300 to 400 mg;

Taurine: from about 50 to 50,000 mg, preferably from about 750 to 8,000 mg, and most preferably from about 3,000 to 5,000 mg;

Melatonin: from about 0.5 to 15 mg, preferably from about 0.5 to 9 mg, and most preferably from about 3 to 5 mg;

Rhodiola Rosea: from about 50 to 20,000 mg, preferably from about 400 to 3,000 mg, and most preferably from about 750 to 1,500 mg;

SAMe: from about 50 to 2,400 mg, preferably from about 200 to 1,200 mg, and most preferably from about 400 to 800 mg;

Glutamine: from about 500 to 8,000 mg, preferably from about 2,000 to 6,000 mg, and most preferably from about 2,000 to about 4,000 mg;

Niacin: from about 100 to 3,000 mg, preferably from about 1,000 to 2,800 mg, and most preferably from about 1,800 to 2,400 mg;

Hops: from about 100 to 1500 mg, preferably from about 400 to 1,200 mg, and most preferably from about 600 to 1,200 mg.

It is very important that the components be provided in their most effective form, as many may be obtained on the market in forms that have reduced, ineffective or unavailable active ingredients, which are due to many factors, including but not limited to the time of harvest, the freshness of the component and storage temperature. In recent years the more popular components of those above have come to be marketed in what is referred to as “standardized” form, which means that the most active ingredient is artificially enhanced to approximate the amount or effectiveness of the active ingredient available in well-prepared fresh herbs or other source plants. The combined standardized form is often far more effective than just the accepted most active ingredients. The difference is commonly accepted to be the synergistic effects of known or unknown less important active ingredients. Thus, for certain components it is important to ascertain the true amount of the most well known active ingredient, as opposed for example to the beneficial and effective amounts set forth above, which correlate to the estimated amount of natural fresh herbs.

Thus, in terms of daily efficacious amounts, for Kudzu, there should be from about 1 to 25 mg of the most active ingredient, preferably from about 3 to 9 mg, and most preferably from about 5 to 7 mg. For St John's Wort, there should be from about 0.9 to 18 mg of the most active ingredient, preferably from about 1.8 to 14 mg, and most preferably from about 5 to 11 mg.

In addition to the above combinations of components, other components may be provided alone or in combination to address specific problems or deficiencies. In certain instances one or more blood sugar stabilizer components are needed, the blood sugar component being chosen from the group of blood sugar stabilizer components consisting of Niacin and Chromium Picolinate. The effective daily dosage amount of Chromium Picolinate is from about 100 to 1,000 mcg, preferably from about 400 to 700 mcg, and most preferably from about 500 to 600 mcg.

Likewise, one or more energizer components may be needed, and the energizer component is chosen from the group of energizer components consisting of Glutamine and Taurine.

Furthermore, one or more organ repair components may be needed to address damaged organs, the organ repair component being chosen from the group of organ repair components consisting of Glutamine, Milk Thistle (Silymarin), Niacin and Taurine. The effective daily dosage amount of Milk Thistle is from about 60 to 1,900 mg, preferably from about 375 to 1,400 mg, and most preferably from about 875 to 1,125 mg. For Milk Thistle, there should be from about 50 to 1500 mg of the most active ingredient, preferably from about 300 to 1,100 mg, and most preferably from about 700 to 900 mg.

The various embodiments of the inventive composition described above have proven to be highly effective in suppressing the addictive elements of alcohol dependency, which stems greatly from the fact that alcohol is a complex molecule that satisfies complex needs for feelings of pleasure, i.e., needs that result from combinations of nutritional, physiological and psychological deficiencies. The individual components described above each have beneficial and efficacious effects on specific nutritional, physiological and psychological deficiencies, but are not sufficient when administered individually to combat the addictive nature of alcohol. It is the combination of components to address diverse needs that successfully treats the addictive nature of alcohol, such that an individual's craving for alcohol is overcome.

It is contemplated that equivalent and substitutions for certain components set forth above may be obvious to those of ordinary skill in the art, and therefore the true scope and definition of the invention is to be asset forth in the following claims.

I claim:

1. A composition effective in the treatment of alcohol dependency, comprising in combination:
an alcohol craving blocking component and
an antidepressant component;
each such component present in beneficially sufficient amount such that the combination is effective to minimize or obviate the nutritional, physiological and psychological conditions that result in alcohol dependency.

2. The composition of claim 1, wherein said alcohol craving blocking component is chosen from the group of alcohol craving blocking components consisting of Kudzu and Rhodiola Rosea.

3. The composition of claim 1, wherein said antidepressant component is chosen from the group of components consisting of St. John's Wort, 5-Hydroxytryptophanm SAMe, Melatonin and Taurine.

4. The composition of claim 2, wherein said antidepressant component is chosen from the group of components consisting of St. John's Wort, 5-Hydroxytryptophanm SAMe, Melatonin and Taurine.

5. The composition of claim 1, further comprising an anti-anxiety component.

6. The composition of claim 5, wherein said anti-anxiety component is chosen from the group of anti-anxiety components consisting of Vitamin B complex, Glutamine, Niacin, SAMe, Hops, Melatonin and Rhodiola Rosea.

7. The composition of claim 2, further comprising an anti-anxiety component, wherein said anti-anxiety component is chosen from the group of anti-anxiety components consisting of Vitamin B complex, Glutamine, Niacin, SAMe, Hops, Melatonin and Rhodiola Rosea.

8. The composition of claim 3, further comprising an anti-anxiety component, wherein said anti-anxiety component is chosen from the group of anti-anxiety components consisting of Vitamin B complex, Glutamine, Niacin, SAMe, Hops, Melatonin and Rhodiola Rosea.

9. The composition of claim 4, further comprising an anti-anxiety component, wherein said anti-anxiety component is chosen from the group of anti-anxiety components consisting of Vitamin B complex, Glutamine, Niacin, SAMe, Hops, Melatonin and Rhodiola Rosea.

10. The composition of claim 1, further comprising a blood sugar stabilizer component.

11. The composition of claim 10, wherein said blood sugar stabilizer component is chosen from the group of blood sugar stabilizer components consisting of Niacin and Chromium Picolinate.

12. The composition of claim 2, further comprising a blood sugar stabilizer component, wherein said blood sugar stabilizer component is chosen from the group of blood sugar stabilizer components consisting of Niacin and Chromium Picolinate.

13. The composition of claim 3, further comprising a blood sugar stabilizer component, wherein said blood sugar stabilizer component is chosen from the group of blood sugar stabilizer components consisting of Niacin and Chromium Picolinate.

14. The composition of claim 4, further comprising a blood sugar stabilizer component, wherein said blood sugar stabilizer component is chosen from the group of blood sugar stabilizer components consisting of Niacin and Chromium Picolinate.

15. The composition of claim 6, further comprising a blood sugar stabilizer component, wherein said blood sugar stabilizer component is chosen from the group of blood sugar stabilizer components consisting of Niacin and Chromium Picolinate.

16. The composition of claim 1, further comprising an energizer component.

17. The composition of claim 16, wherein said energizer component is chosen from the group of energizer components consisting of Glutamine and Taurine.

18. The composition of claim 2, further comprising an energizer component, wherein said energizer component is chosen from the group of energizer components consisting of Glutamine and Taurine.

19. The composition of claim 3, further comprising an energizer component, wherein said energizer component is chosen from the group of energizer components consisting of Glutamine and Taurine.

20. The composition of claim 4, further comprising an energizer component, wherein said energizer component is chosen from the group of energizer components consisting of Glutamine and Taurine.

21. The composition of claim 6, further comprising an energizer component, wherein said energizer component is chosen from the group of energizer components consisting of Glutamine and Taurine.

22. The composition of claim 11, further comprising an energizer component, wherein said energizer component is chosen from the group of energizer components consisting of Glutamine and Taurine.

23. The composition of claim 1, further comprising an organ repair component.

24. The composition of claim 23, wherein said organ repair component being chosen from the group of organ repair components consisting of Glutamine, Milk Thistle, Niacin and Taurine.

25. The composition of claim 2, further comprising an organ repair component, wherein said organ repair component being chosen from the group of organ repair components consisting of Glutamine, Milk Thistle, Niacin and Taurine.

26. The composition of claim 3, further comprising an organ repair component, wherein said organ repair component being chosen from the group of organ repair components consisting of Glutamine, Milk Thistle, Niacin and Taurine.

27. The composition of claim 4, further comprising an organ repair component, wherein said organ repair component being chosen from the group of organ repair components consisting of Glutamine, Milk Thistle, Niacin and Taurine.

28. The composition of claim 6, further comprising an organ repair component, wherein said organ repair component being chosen from the group of organ repair components consisting of Glutamine, Milk Thistle, Niacin and Taurine.

29. The composition of claim 11, further comprising an organ repair component, wherein said organ repair component being chosen from the group of organ repair components consisting of Glutamine, Milk Thistle, Niacin and Taurine.

30. The composition of claim 17, further comprising an organ repair component, wherein said organ repair component being chosen from the group of organ repair components consisting of Glutamine, Milk Thistle, Niacin and Taurine.

31. A composition effective in reducing or obviating alcohol addiction when administered on a daily basis, said composition comprising:
from about 450 to 11,500 mg of Kudzu and
from about 300 to 6,000 mg of St. John's Wort.

32. The composition of claim 31, wherein said Kudzu is present from about 1,350 to 4,100 mg.

33. The composition of claim 31, wherein said Kudzu is present from about 2,300 to 3,200 mg.

34. The composition of claim 31, further comprising Vitamin B complex.

35. A composition effective in reducing or obviating alcohol addiction when administered on a daily basis, said composition comprising from about 450 to 11,500 mg of Kudzu and Vitamin B complex.

36. A composition effective in reducing or obviating alcohol addiction when administered on a daily basis, said composition comprising:
from about 450 to 11,500 mg of Kudzu and
from about 25 to 800 mg of 5-Hydroxytryptophan.

37. The composition of claim 36, wherein said Kudzu is present from about 1,350 to 4,100 mg and wherein said 5-Hydroxytryptophan is present from about 100 to 500 mg.

38. The composition of claim 36, wherein said Kudzu is present from about 2,300 to 3,200 mg and wherein said 5-Hydroxytryptophan is present from about 300 to 400 mg.

39. The composition of claim 36, further comprising Vitamin B complex.

40. A composition effective in reducing or obviating alcohol addiction when administered on a daily basis, said composition comprising:
from about 450 to 11,500 mg of Kudzu and
from about 50 to 15,000 mg of Taurine.

41. The composition of claim 40, wherein said Kudzu is present from about 1,350 to 4,100 mg and wherein said Taurine is present from about 750 to 8,000 mg.

42. The composition of claim 40, wherein said Kudzu is present from about 2,300 to 43,200 mg and wherein said Taurine is present from about 3,000 to 5,000 mg.

43. The composition of claim 40, further comprising Vitamin B complex.

44. A composition effective in reducing or obviating alcohol addiction when administered on a daily basis, said composition comprising:
from about 450 to 11,500 mg of Kudzu and
from about 0.5 to 15 mg of Melatonin.

45. The composition of claim 44, wherein said Kudzu is present from about 1,350 to 4,100 mg and wherein said Melatonin is present from about 0.5 to 9 mg.

46. The composition of claim 44, wherein said Kudzu is present from about 2,300 to 3,200 mg and wherein said Melatonin is present from about 3 to 5 mg.

47. The composition of claim 44, further comprising Vitamin B complex.
